(19) United States
(12) Patent Application Publication
(10) Pub. No.: US 2014/0034880 A1
(43) Pub. Date: Feb. 6, 2014

(54) CONJUGATED POLYMERS

(71) Applicants: Nicolas Blouin, Southampton (GB); William Mitchell, Chandler's Ford (GB); Amy Topley, Southampton (GB); Steven Tierney, Southampton (GB)

(72) Inventors: Nicolas Blouin, Southampton (GB); William Mitchell, Chandler's Ford (GB); Amy Topley, Southampton (GB); Steven Tierney, Southampton (GB)

(73) Assignee: MERCK PATENT GMBH, Darmstadt (DE)

(21) Appl. No.: 14/112,313

(22) PCT Filed: Mar. 26, 2012
(86) PCT No.: PCT/EP2012/001321

§ 371 Date: Oct. 17, 2013

(30) Foreign Application Priority Data
Apr. 18, 2011 (EP) 11003231.5

(51) Int. Cl. H01L 51/00; C08G 75/32

(52) U.S. Cl. CPC H01L 51/004; C08G 75/32
USPC 252/500; 528/380; 524/592; 524/542; 548/153

(57) ABSTRACT

The invention relates to novel benzo[1,2-b:4,5-b′]dithiophene-2,6-diyl-alt-thieno[3,4-b]thiazole-4,6-diyl polymers, methods for their preparation and monomers used therein, blends, mixtures and formulations containing them, the use of the polymers, blends, mixtures and formulations as semiconductor in organic electronic (OE) devices, especially in organic photovoltaic (OPV) devices, and to OE and OPV devices comprising these polymers, blends, mixtures or formulations.

### TECHNICAL FIELD

The invention relates to novel benzo[1,2-b:4,5-b′]dithiophene-2,6-diyl-alt-thieno[3,4-b]thiazole-4,6-diyl polymers, methods for their preparation and monomers used therein, blends, mixtures and formulations containing them, the use of the polymers, blends, mixtures and formulations as semiconductor in organic electronic (OE) devices, especially in organic photovoltaic (OPV) devices, and to OE and OPV devices comprising these polymers, blends, mixtures or formulations.

### BACKGROUND

In recent years there has been growing interest in the use of conjugated, semiconducting polymers for electronic applications. One particular area of importance is organic photovoltaics (OPV). Conjugated polymers have found use in OPVs as they allow devices to be manufactured by solution-processing techniques such as spin casting, dip coating or ink jet printing. Solution processing can be carried out cheaper and on a larger scale compared to the evaporative techniques used to make inorganic thin film devices. Currently, polymer based photovoltaic devices are achieving efficiencies up to 8%.

The conjugated polymer serves as the main absorber of the solar energy, therefore a low band gap is a basic requirement of the ideal polymer design to absorb the maximum of the solar spectrum. A commonly used strategy to provide conjugated polymers with narrow band gap is to utilize alternating copolymers consisting of both electron rich donor units and electron deficient acceptor units within the polymer backbone.

However, the conjugated polymers that have been suggested in prior art for use ion OPV devices do still suffer from certain drawbacks. For example many polymers suffer from limited solubility in commonly used organic solvents, which can inhibit their suitability for device manufacturing methods based on solution processing, or show only limited power conversion efficiency in OPV bulk-hetero-junction devices, or have only limited charge carrier mobility, or are difficult to synthesize and require synthesis methods which are unsuitable for mass production.

Therefore, there is still a need for organic semiconducting (OSC) materials that are easy to synthesize, especially by methods suitable for mass production, show good structural organization and film-forming properties, exhibit good electronic properties, especially a high charge carrier mobility, good processibility, especially a high solubility in organic solvents, and high stability in air. Especially for use in OPV cells, there is a need for OSC materials having a low bandgap, which enable improved light harvesting by the photoactive layer and can lead to higher cell efficiencies, compared to the polymers from prior art.

It was an aim of the present invention to provide compounds for use as organic semiconducting materials that do not have the drawbacks of prior art materials as described above, are easy to synthesize, especially by methods suitable for mass production, and do especially show good processibility, high stability, good solubility in organic solvents, high charge carrier mobility, and a low bandgap. Another aim of the invention was to extend the pool of OSC materials available to the expert. Other aims of the present invention are immediately evident to the expert from the following detailed description.

The inventors of the present invention have found that one or more of the above aims can be achieved by providing conjugated alternating copolymers of benzo[1,2-b:4,5-b′]dithiophene-4,6-diyl and thieno[3,4-b]thiazole-4,6-diyl units which are preferably substituted by alkyl, fluoroalkyl, keto or ester groups.

Polymers comprising thieno[3,4-d]thiazole-4,6-diyl units are disclosed in US 2008/0200634 A1 and Bull. Korean Chem. Soc. 2007, 28, 2511-2513. However, these documents do not disclose the alternating copolymers of the present invention.

It was found that conjugated polymers as claimed according to the present invention show good processability and high solubility in organic solvents, and are thus especially suitable for large scale production using solution processing methods. At the same time, they show a low bandgap, high charge carrier mobility, high external quantum efficiency in BHJ solar cells, good morphology when used in p/n-type blends e.g. with fullerenes, high oxidative stability, and are promising materials for organic electronic OE devices, especially for OPV devices with high power conversion efficiency.

### SUMMARY

The invention relates to conjugated polymers of the following formula

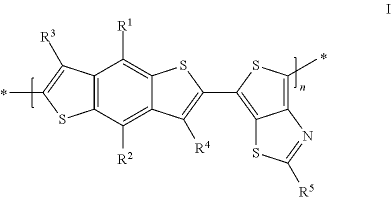

wherein
R¹ to R⁵ denote independently of each other, and on each occurrence identically or differently, H, halogen, or an optionally substituted carbyl or hydrocarbyl group, wherein one or more C atoms are optionally replaced by a hetero atom, and
n is an integer >1.

The invention further relates to monomers suitable for the preparation of polymers of formula I.

The invention further relates to the use of the polymers of formula I as p-type semiconductor.

The invention further relates to the use of the polymers according to the present invention as electron donor component in semiconducting materials, formulations, polymer blends, devices or components of devices.

The invention further relates to a semiconducting material, formulation, polymer blend, device or component of a device comprising a polymer of formula I as electron donor component, and preferably further comprising one or more compounds or polymers having electron acceptor properties.

The invention further relates to a mixture or polymer blend comprising one or more polymers according to the present invention and one or more additional compounds or polymers which are preferably selected from compounds and polymers having one or more of semiconducting, charge transport, hole or electron transport, hole or electron blocking, electrically conducting, photoconducting or light emitting properties.

The invention further relates to a mixture or polymer blend as described above and below, which comprises one or more polymers according to of the present invention and one or more n-type organic semiconductor compounds, preferably selected from fullerenes or substituted fullerenes.

The invention further relates to a formulation comprising one or more polymers, mixtures or polymer blends according to the present invention and optionally one or more solvents, preferably selected from organic solvents.

The invention further relates to the use of polymers, mixtures, polymer blends and formulations according to the present invention as charge transport, semiconducting, electrically conducting, photoconducting or light emitting material in an optical, electrooptical, electronic, electroluminescent or photoluminescent device, or in a component of such a device, or in an assembly comprising such a device or component.

The invention further relates to a charge transport, semiconducting, electrically conducting, photoconducting or light emitting material or component comprising one or more polymers, mixtures, polymer blends or formulations according to the present invention.

The invention further relates to an optical, electrooptical, electronic, electroluminescent or photoluminescent device, or a component thereof, or an assembly comprising it, which comprises one or more polymers, mixtures, polymer blends or formulations according to the present invention, or comprises a charge transport, semiconducting, electrically conducting, photoconducting or light emitting material according to the present invention.

The optical, electrooptical, electronic, electroluminescent and photoluminescent devices include, without limitation, organic field effect transistors (OFETs), organic thin film transistors (OTFTs), organic light emitting diodes (OLEDs), organic light emitting transistors (OLETs), organic photovoltaic devices (OPVs), organic solar cells, laser diodes, organic plasmon-emitting diodes (OPEDs), Schottky diodes, organic photoconductors (OPCs) and organic photodetectors (OPDs).

The components of the above devices include, without limitation, charge injection layers, charge transport layers, interlayers, planarising layers, antistatic films, polymer electrolyte membranes (PEMs), conducting substrates and conducting patterns.

The assemblies comprising such devices or components include, without limitation, integrated circuits (ICs), radio frequency identification (RFID) tags or security markings or security devices containing them, flat panel displays or backlights thereof, electrophotographic devices, electrophotographic recording devices, organic memory devices, sensor devices, biosensors and biochips.

In addition the compounds, polymers, mixtures, polymer blends and formulations of the present invention can be used as electrode materials in batteries and in components or devices for detecting and discriminating DNA sequences.

### DETAILED DESCRIPTION

The monomers and polymers of the present invention are easy to synthesize and exhibit several advantageous properties, like a low bandgap, a high charge carrier mobility, a high solubility in organic solvents, a good processability for the device manufacture process, a high oxidative stability and a long lifetime in electronic devices.

The unit of formula I is especially suitable as (electron) donor unit in p-type semiconducting polymers or copolymers, in particular copolymers containing both donor and acceptor units, and for the preparation of blends of p-type and n-type semiconductors which are useful for application in bulk heterojunction photovoltaic devices.

In addition, they show the following advantageous properties:
i) The 4,6-dibromo-thieno[3,4-d]thiazole monomers exhibit better thermal, light and air stability compared for example to 4,6-dibromo-thieno[3,4-b]thiophene monomers.
ii) The additional nitrogen atom on the thieno[3,4-d]thiazole fused ring will lower the resulting polymer LUMO energy level, therefore lowers the band gap of the resultant polymer, improving the light harvesting ability of the material.
iii) Additional solubility can be introduced into the polymer by inclusion at the terminal R1, R2, R3, R4 or R5 positions of solubilising groups.
iv) Additional fine-tuning of the electronic energies (HOMO/LUMO levels) by either careful selection of thiazole R5 group on each side or benzo[1,2-b;4,5-b′]dithiophene R1 and R2 group should afford candidate materials for organic photovoltaic applications.

The synthesis of the polymer of formula I and its corresponding monomer can be achieved based on methods that are known to the skilled person and described in the literature, as will be further illustrated herein.

Above and below, the term “polymer” generally means a molecule of high relative molecular mass, the structure of which essentially comprises the multiple repetition of units derived, actually or conceptually, from molecules of low relative molecular mass (PAC, 1996, 68, 2291). The term “oligomer” generally means a molecule of intermediate relative molecular mass, the structure of which essentially comprises a small plurality of units derived, actually or conceptually, from molecules of lower relative molecular mass (PAC, 1996, 68, 2291). In a preferred sense according to the present invention a polymer means a compound having >1, i.e. at least 2 repeating units, preferably ≧5 repeating units, and an oligomer means a compound with >1 and <10, preferably <5, repeating units.

Above and below, in a formula showing a polymer or a repeating unit, like formula I and its subformulae, an asterisk (“*”) denotes a linkage to an adjacent repeating unit or a terminal group in the polymer chain.

The terms “repeating unit” and “monomeric unit” mean the constitutional repeating unit (CRU), which is the smallest constitutional unit the repetition of which constitutes a regular macromolecule, a regular oligomer molecule, a regular block or a regular chain (PAC, 1996, 68, 2291).

The terms “donor” and “acceptor”, unless stated otherwise, mean an electron donor or electron acceptor, respectively. “Electron donor” means a chemical entity that donates electrons to another compound or another group of atoms of a compound. “Electron acceptor” means a chemical entity that accepts electrons transferred to it from another compound or another group of atoms of a compound. (see also U.S. Environmental Protection Agency, 2009, Glossary of technical terms, http://www.epa.gov/oust/cat/TUMGLOSS.HTM).

The term “leaving group” means an atom or group (charged or uncharged) that becomes detached from an atom in what is considered to be the residual or main part of the molecule taking part in a specified reaction (see also PAC, 1994, 66, 1134).

The term “conjugated” means a compound containing mainly C atoms with sp²-hybridisation (or optionally also sp-hybridisation), which may also be replaced by hetero atoms. In the simplest case this is for example a compound with alternating C—C single and double (or triple) bonds, but does also include compounds with units like 1,3-phenylene. “Mainly” means in this connection that a compound with naturally (spontaneously) occurring defects, which may lead to interruption of the conjugation, is still regarded as a conjugated compound.

Unless stated otherwise, the molecular weight is given as the number average molecular weight Mₙ or weight average molecular weight M_{W}, which is determined by gel permeation chromatography (GPC) against polystyrene standards in eluent solvents such as tetrahydrofuran, trichloromethane (TCM, chloroform), chlorobenzene or 1,2,4-trichlorobenzene. Unless stated otherwise, 1,2,4-trichlorobenzene is used as solvent. The degree of polymerization, also referred to as total number of repeating units, n, means the number average degree of polymerization given as n=Mₙ/M_{U}, wherein Mₙ is the number average molecular weight and M_{U} is the molecular weight of the single repeating unit, see J. M. G. Cowie, Polymers: Chemistry & Physics of Modern Materials, Blackie, Glasgow, 1991.

The term “carbyl group” as used above and below denotes any monovalent or multivalent organic radical moiety which comprises at least one carbon atom either without any non-carbon atoms (like for example —C≡C—), or optionally combined with at least one non-carbon atom such as N, O, S, P, Si, Se, As, Te or Ge (for example carbonyl etc.). The term “hydrocarbyl group” denotes a carbyl group that does additionally contain one or more H atoms and optionally contains one or more hetero atoms like for example N, O, S, P, Si, Se, As, Te or Ge.

The term “hetero atom” means an atom in an organic compound that is not a H- or C-atom, and preferably means N, O, S, P, Si, Se, As, Te or Ge.

A carbyl or hydrocarbyl group comprising a chain of 3 or more C atoms may be straight-chain, branched and/or cyclic, including spiro and/or fused rings.

Preferred carbyl and hydrocarbyl groups include alkyl, alkoxy, alkylcarbonyl, alkoxycarbonyl, alkylcarbonyloxy and alkoxycarbonyloxy, each of which is optionally substituted and has 1 to 40, preferably 1 to 25, very preferably 1 to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 6 to 40, preferably 7 to 40 C atoms, wherein all these groups do optionally contain one or more hetero atoms, preferably selected from N, O, S, P, Si, Se, As, Te and Ge.

The carbyl or hydrocarbyl group may be a saturated or unsaturated acyclic group, or a saturated or unsaturated cyclic group. Unsaturated acyclic or cyclic groups are preferred, especially aryl, alkenyl and alkynyl groups (especially ethynyl). Where the C₁-C₄₀ carbyl or hydrocarbyl group is acyclic, the group may be straight-chain or branched. The C₁-C₄₀ carbyl or hydrocarbyl group includes for example: a C₁-C₄₀ alkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₂-C₂₀ alkenyl group, a C₂-C₂₀ alkynyl group, a C₃-C₂₀ allyl group, a C₄-C₂₀ alkyldienyl group, a C₆-C₁₂ aryl group, and a C₄-C₂₀ polyenyl group, respectively. Also included are combinations of groups having carbon atoms and groups having hetero atoms, like e.g. an alkynyl group, preferably ethynyl, that is substituted with a silyl group, preferably a trialkylsilyl group.

Aryl and heteroaryl preferably denote a mono-, bi- or tricyclic aromatic or heteroaromatic group with 4 to 30 ring C atoms that may also comprise condensed rings and is optionally substituted with one or more groups L,

wherein L is selected from halogen, —CN, —NC, —NCO, —NCS, —OCN, —SCN, —C(═O)NR⁰R⁰⁰, —C(═O)X⁰, —C(═O)R⁰, —NH₂, —NR⁰R⁰⁰, —SH, —SR⁰, —SO₃H, —SO₂R⁰, —OH, —NO₂, —CF₃, —SF₅, P-Sp-, optionally substituted silyl, or carbyl or hydrocarbyl with 1 to 40 C atoms that is optionally substituted and optionally comprises one or more hetero atoms, and is preferably alkyl, alkoxy, thiaalkyl, alkylcarbonyl, alkoxycarbonyl or alkoxycarbonyloxy with 1 to 20 C atoms that is optionally fluorinated, and R⁰, R⁰⁰, X⁰, P and Sp have the meanings given above and below.

Very preferred substituents L are selected from halogen, most preferably F, or alkyl, alkoxy, oxaalkyl, thioalkyl, fluoroalkyl and fluoroalkoxy with 1 to 12 C atoms or alkenyl, alkynyl with 2 to 12 C atoms.

Especially preferred aryl and heteroaryl groups are phenyl in which, in addition, one or more CH groups may be replaced by N, naphthalene, thiophene, selenophene, thienothiophene, dithienothiophene, fluorene and oxazole, all of which can be unsubstituted, mono- or polysubstituted with L as defined above. Very preferred rings are selected from pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene preferably 2-thiophene, selenophene, preferably 2-selenophene, thieno[3,2-b]thiophene, indole, isoindole, benzofuran, benzothiophene, benzodithiophene, quinole, 2-methylquinole, isoquinole, quinoxaline, quinazoline, benzotriazole, benzimidazole, benzothiazole, benzisothiazole, benzisoxazole, benzoxadiazole, benzoxazole, benzothiadiazole, all of which can be unsubstituted, mono- or polysubstituted with L as defined above. Further examples of heteroaryl groups are those selected from the following formulae

An alkyl or alkoxy radical, i.e. where the terminal CH₂ group is replaced by —O—, can be straight-chain or branched. It is preferably straight-chain, has 2, 3, 4, 5, 6, 7 or 8 carbon atoms and accordingly is preferably ethyl, propyl, butyl, pentyl, hexyl, heptyl, octyl, ethoxy, propoxy, butoxy, pentoxy, hexoxy, heptoxy, or octoxy, furthermore methyl, nonyl, decyl, undecyl, dodecyl, tridecyl, tetradecyl, pentadecyl, nonoxy, decoxy, undecoxy, dodecoxy, tridecoxy or tetradecoxy, for example.

An alkenyl group, wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example. Oxaalkyl, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

In an alkyl group wherein one CH₂ group is replaced by —O— and one by —C(O)—, these radicals are preferably neighboured. Accordingly these radicals together form a carbonyloxy group —C(O)—O— or an oxycarbonyl group —O—C(O)—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxyethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxycarbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)propyl, 3-(ethoxycarbonyl)propyl, 4-(methoxycarbonyl)-butyl.

An alkyl group wherein two or more CH₂ groups are replaced by —O— and/or —C(O)O— can be straight-chain or branched. It is preferably straight-chain and has 3 to 12 C atoms. Accordingly it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

A thioalkyl group, i.e where one CH₂ group is replaced by —S—, is preferably straight-chain thiomethyl (—SCH₃), 1-thioethyl (—SCH₂CH₃), 1-thiopropyl (═—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridised vinyl carbon atom is replaced.

A fluoroalkyl group is preferably straight-chain perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or C₈F₁₇, very preferably C₆F₁₃.

The above-mentioned alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 3-methylpentyl, 2-ethylhexyl, 2-propylpentyl, in particular 2-methylbutyl, 2-methylbutoxy, 2-methylpentoxy, 3-methylpentoxy, 2-ethylhexoxy, 1-methylhexoxy, 2-octyloxy, 2-oxa-3-methylbutyl, 3-oxa-4-methylpentyl, 4-methylhexyl, 2-hexyl, 2-octyl, 2-nonyl, 2-decyl, 2-dodecyl, 6-methoxyoctoxy, 6-methyloctoxy, 6-methyloctanoyloxy, 5-methylheptyloxy-carbonyl, 2-methylbutyryloxy, 3-methylvaleroyloxy, 4-methylhexanoyloxy, 2-chloropropionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxahexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example. Very preferred are 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tert. butyl, isopropoxy, 2-methyl-propoxy and 3-methylbutoxy.

In another preferred embodiment of the present invention, R¹⁻⁵ are independently of each other selected from primary, secondary or tertiary alkyl or alkoxy with 1 to 30 C atoms, wherein one or more H atoms are optionally replaced by F, or aryl, aryloxy, heteroaryl or heteroaryloxy that is optionally alkylated or alkoxylated and has 4 to 30 ring atoms. Very preferred groups of this type are selected from the group consisting of the following formulae

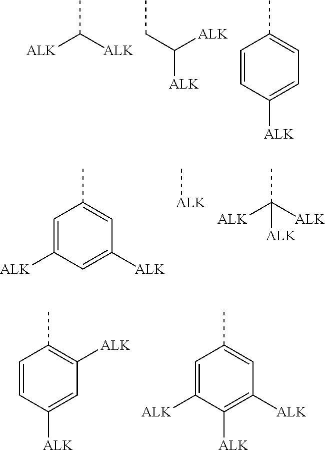

wherein “ALK” denotes optionally fluorinated, preferably linear, alkyl or alkoxy with 1 to 20, preferably 1 to 12 C-atoms, in case of tertiary groups very preferably 1 to 9 C atoms, and the dashed line denotes the link to the ring to which these groups are attached. Especially preferred among these groups are those wherein all ALK subgroups are identical.

—CY¹═CY²— is preferably —CH═CH—, —CF═CF— or —CH═C(CN)—.

Halogen is F, Cl, Br or I, preferably F, Cl or Br.

—CO—, —C(═O)— and —C(O)— denote a carbonyl group, i.e.

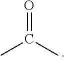

The units and polymers may also be substituted with a polymerisable or crosslinkable reactive group, which is optionally protected during the process of forming the polymer. Particular preferred units polymers of this type are those comprising one or more units of formula I wherein one or more of R¹⁻⁴ denote or contain a group P-Sp-. These units and polymers are particularly useful as semiconductors or charge transport materials, as they can be crosslinked via the groups P, for example by polymerisation in situ, during or after processing the polymer into a thin film for a semiconductor component, to yield crosslinked polymer films with high charge carrier mobility and high thermal, mechanical and chemical stability.

Preferably the polymerisable or crosslinkable group P is selected from CH₂═CW¹—C(O)—O—, CH₂═CW¹—C(O)—,

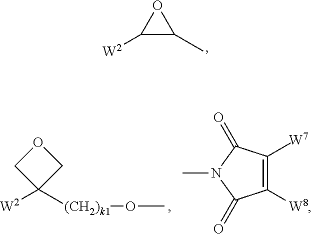

CH₂═CW²—(O)ₖ₁—, CW¹═CH—C(O)—(O)ₖ₃—, CW¹═CH—C(O)—NH—, CH₂═CW¹—C(O)—NH—, CH₃—CH═CH—O—, (CH₂═CH)₂CH—OC(O)—, (CH₂═CH—CH₂)₂CH—O—C(O)—, (CH₂═CH)₂CH—O—, (CH₂═CH—CH₂)₂N—, (CH₂═CH—CH₂)₂N—C(O)—, HO—CW²W³—, HS—CW²W³—, HW²N—, HO—CW²W³—NH—, CH₂═CH—(C(O)—O)ₖ₁-Phe-(O)ₖ₂—, CH₂═CH—(C(O))ₖ₁-Phe-(O)ₖ₂—, Phe-CH═CH—, HOOC—, OCN—, and W⁴W⁵W⁶Si—, with W¹ being H, F, Cl, CN, CF₃, phenyl or alkyl with 1 to 5 C-atoms, in particular H, Cl or CH₃, W² and W³ being independently of each other H or alkyl with 1 to 5 C-atoms, in particular H, methyl, ethyl or n-propyl, W⁴, W⁵ and W⁶ being independently of each other Cl, oxaalkyl or oxacarbonylalkyl with 1 to 5 C-atoms, W⁷ and W⁸ being independently of each other H, Cl or alkyl with 1 to 5 C-atoms, Phe being 1,4-phenylene that is optionally substituted by one or more groups L as defined above, k₁, k₂ and k₃ being independently of each other 0 or 1, k₃ preferably being 1, and k₄ being an integer from 1 to 10.

Alternatively P is a protected derivative of these groups which is non-reactive under the conditions described for the process according to the present invention. Suitable protective groups are known to the ordinary expert and described in the literature, for example in Green, “Protective Groups in Organic Synthesis”, John Wiley and Sons, New York (1981), like for example acetals or ketals.

Especially preferred groups P are CH₂═CH—C(O)—O—, CH₂═C(CH₃)—C(O)—O—, CH₂═CF—C(O)—O—, CH₂═CH—O—, (CH₂═CH)₂CH—O—C(O)—, (CH₂═CH)₂CH—O—,

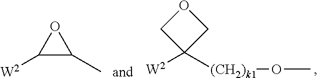

or protected derivatives thereof. Further preferred groups P are selected from the group consisting of vinyloxy, acrylate, methacrylate, fluoroacrylate, chloracrylate, oxetan and epoxy groups, very preferably from an acrylate or methacrylate group.

Polymerisation of group P can be carried out according to methods that are known to the ordinary expert and described in the literature, for example in D. J. Broer; G. Challa; G. N. Mol, Macromol. Chem, 1991, 192, 59.

The term “spacer group” is known in prior art and suitable spacer groups Sp are known to the ordinary expert (see e.g. Pure Appl. Chem. 73(5), 888 (2001). The spacer group Sp is preferably of formula Sp′-X′, such that P-Sp- is P-Sp′-X′-, wherein
Sp′ is alkylene with up to 30 C atoms which is unsubstituted or mono- or polysubstituted by F, Cl, Br, I or CN, it being also possible for one or more non-adjacent CH₂ groups to be replaced, in each case independently from one another, by —O—, —S—, —NH—, —NR⁰—, —SiR⁰R⁰⁰—, —C(O)—, —C(O)O—, —OC(O)—, —OC(O)—O—, —S—C(O)—, —C(O)—S—, —CH═CH— or —C≡C— in such a manner that O and/or S atoms are not linked directly to one another,
X′ is —O—, —S—, —C(O)—, —C(O)O—, —OC(O)—, —O—C(O)O—, —C(O)—NR⁰—, —NR⁰—C(O)—, —NR⁰—C(O)—NR⁰⁰—, —OCH₂—, —CH₂O—, —SCH₂—, —CH₂S—, —CF₂O—, —OCF₂—, —CF₂S—, —SCF₂—, —CF₂CH₂—, —CH₂CF₂—, —CF₂CF₂—, —CH═N—, —N═CH—, —N═N—, —CH═CR⁰—, —CY¹═CY²—, —C≡C—, —CH═CH—C(O)O—, —OC(O)—CH═CH— or a single bond,
R⁰ and R⁰⁰ are independently of each other H or alkyl with 1 to 12 C-atoms, and
Y¹ and Y² are independently of each other H, F, Cl or CN.

X′ is preferably —O—, —S—, —OCH₂—, —CH₂O—, —SCH₂—, —CH₂S—, —CF₂O—, —OCF₂—, —CF₂S—, —SCF₂—, —CH₂CH₂—, —CF₂CH₂—, —CH₂CF₂—, —CF₂CF₂—, —CH═N—, —N═CH—, —N═N—, —CH═CR⁰—, —CY¹═CY²—, —C≡C— or a single bond, in particular —O—, —S—, —C≡C—, —CY¹═CY²— or a single bond. In another preferred embodiment X′ is a group that is able to form a conjugated system, such as —C≡C— or —CY¹═CY²—, or a single bond.

Typical groups Sp′ are, for example, —(CH₂)ₚ—, —(CH₂CH₂O)_{q}—CH₂CH₂—, —CH₂CH₂—S—CH₂CH₂— or —CH₂CH₂—NH—CH₂CH₂— or —(SiR⁰R⁰⁰—O)ₚ—, with p being an integer from 2 to 12, q being an integer from 1 to 3 and R⁰ and R⁰⁰ having the meanings given above.

Preferred groups Sp′ are ethylene, propylene, butylene, pentylene, hexylene, heptylene, octylene, nonylene, decylene, undecylene, dodecylene, octadecylene, ethyleneoxyethylene, methyleneoxybutylene, ethylene-thioethylene, ethylene-N-methyl-iminoethylene, 1-methylalkylene, ethenylene, propenylene and butenylene for example.

In the polymers according to the present invention, the total number of repeating units n is preferably from 2 to 10,000. The total number of repeating units n is preferably ≧5, very preferably ≧10, most preferably ≧50, and preferably ≦500, very preferably ≦1,000, most preferably ≦2,000, including any combination of the aforementioned lower and upper limits of n.

Preferred polymers of formula I are selected of formula I1

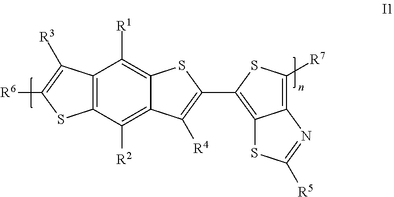

wherein R¹⁻⁵ and n are as defined above and below, and R⁶ and R⁷ have independently of each other one of the meanings of R³ as defined above, or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′, P-Sp- or an endcap group, wherein P and Sp are as defined above, X′ and X″ denote halogen, R′, R″ and R′″ have independently of each other one of the meanings of R⁰ given above, and two of R′, R″ and R′″ may also form a ring together with the hetero atom to which they are attached.

Preferred endcap groups R⁵ and R⁶ are H, C₁₋₂₀ alkyl, or optionally substituted C₆₋₁₂ aryl or C₂₋₁₀ heteroaryl, very preferably H or phenyl.

Another aspect of the invention relates to monomers of formula II

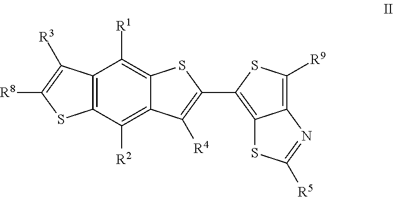

wherein R¹⁻⁵ are as defined above and below, and R⁸ and R⁹ are, preferably independently of each other, selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, preferably Cl, Br or I, Z¹⁻⁴ are selected from the group consisting of alkyl and aryl, each being optionally substituted, and two groups Z² may also together form a cyclic group.

Preferably R¹, R², R³, R⁴ and R⁵ in formula I, I1 and II denote independently of each other F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms, or P-Sp-, wherein
R⁰ and R⁰⁰ are independently of each other H or optionally substituted C₁₋₄₀ carbyl or hydrocarbyl, preferably H or alkyl with 1 to 12 C atoms,
P is a polymerisable or crosslinkable group,
Sp is a spacer group or a single bond,
X⁰ is halogen, preferably F, Cl or Br,

In preferred polymers and monomers of formula I, I1 and II R¹, R² and R⁵ denote independently of each other, and on each occurrence identically or differently, straight-chain, branched or cyclic alkyl with 1 to 30 C atoms, preferably 1 to 20 C atoms, in which one or more non-adjacent C atoms are optionally replaced by —O—, —S—, —C(O)—, —C(O)—O—, —O—C(O)—, —CH═CH— or —C≡C— and which are unsubstituted or substituted by F, Cl, Br, I or CN, and preferably R³ and R⁴ are H.

In very preferred polymers and monomers of formula I, I1 and II R¹, R² and/or R⁵ denote independently of each other straight-chain or branched alkyl with 1 to 20 C atoms which is unsubstituted or substituted by one or more F atoms, or alkylcarbonyl, alkoxycarbonyl or alkylcarbonyloxy with 2 to 20 C atoms, and preferably R³ and R⁴ are H.

Preferably R³ and R⁴ in formula I, I1 and II denote H.

Further preferred are polymers and monomers of formula I, I1 and II selected from the following list of preferred embodiments:
n is at least 5, preferably at least 10, very preferably at least 50, and up to 2,000, preferably up to 500.
M_{w} is at least 5,000, preferably at least 8,000, very preferably at least 10,000, and preferably up to 300,000, very preferably up to 100,000,
R¹ and R² are independently of each other selected from the group consisting of primary alkyl or alkoxy with 1 to 30 C atoms, secondary alkyl or alkoxy with 3 to 30 C atoms, and tertiary alkyl or alkoxy with 4 to 30 C atoms, wherein in all these groups one or more H atoms are optionally replaced by F,
R¹ and R² are independently of each other selected from the group consisting of aryl, heteroaryl, aryloxy, heteroaryloxy, each of which is optionally alkylated or alkoxylated and has 4 to 30 ring atoms,
R¹ and/or R² are independently of each other selected from the group consisting of alkyl, alkoxy, alkylcarbonyl, alkoxycarbonyl and alkylcarbonyloxy, all of which are straight-chain or branched, are optionally fluorinated, and have from 1 to 30 C atoms, and aryl, aryloxy, heteroaryl and heteroaryloxy, all of which are optionally alkylated or alkoxylated and have 4 to 30 ring atoms,
R¹ and R² denote independently of each other F, Cl, Br, I, CN, R¹⁰, —C(O)—R¹⁰, —C(O)—O—R¹⁰, or —O—C(O)—R¹⁰, wherein R¹⁰ is straight-chain, branched or cyclic alkyl with 1 to 30 C atoms, in which one or more non-adjacent C atoms are optionally replaced by —O—, —S—, —C(O)—, —C(O)—O—, —O—C(O)—, —O—C(O)—O—, —CR⁰═CR⁰⁰— or —C≡C— and in which one or more H atoms are optionally replaced by F, Cl, Br, I or CN, or R¹⁰ is aryl or heteroaryl having 4 to 30 ring atoms which is unsubstituted or which is substituted by one or more halogen atoms or by one or more groups R¹ as defined above,
R¹ and/or R² denote independently of each other aryl, aryloxy, heteroaryl or heteroaryloxy having 4 to 30 ring atoms which is unsubstituted or which is substituted by one or more halogen atoms or by one or more groups R¹⁰, —C(O)—R¹⁰, —C(O)—O—R¹⁰, or —O—C(O)—R¹⁰ as defined above,
R⁵ is selected from the group consisting of primary alkyl or alkoxy with 1 to 30 C atoms, secondary alkyl or alkoxy with 3 to 30 C atoms, and tertiary alkyl or alkoxy with 4 to 30 C atoms, wherein in all these groups one or more H atoms are optionally replaced by F,
R⁵ is selected from the group consisting of aryl, heteroaryl, aryloxy, heteroaryloxy, each of which is optionally alkylated or alkoxylated and has 4 to 30 ring atoms,
R⁵ is selected from the group consisting of alkyl, alkoxy, alkylcarbonyl, alkoxycarbonyl and alkylcarbonyloxy, all of which are straight-chain or branched, are optionally fluorinated, and have from 1 to 30 C atoms, and aryl, aryloxy, heteroaryl and heteroaryloxy, all of which are optionally alkylated or alkoxylated and have 4 to 30 ring atoms,
R⁵ denotes F, Cl, Br, I, CN, R¹⁰, —C(O)—R¹⁰, —C(O)—O—R¹⁰, or —O—C(O)—R¹⁰, wherein R¹⁰ is straight-chain, branched or cyclic alkyl with 1 to 30 C atoms, in which one or more non-adjacent C atoms are optionally replaced by —O—, —S—, —C(O)—, —C(O)—O—, —O—C(O)—, —O—C(O)—O—, —CR⁰═CR⁰⁰— or —C≡C— and in which one or more H atoms are optionally replaced by F, Cl, Br, I or CN, or R¹⁰ is aryl or heteroaryl having 4 to 30 ring atoms which is unsubstituted or which is substituted by one or more halogen atoms or by one or more groups R¹ as defined above,
R⁵ denotes aryl, aryloxy, heteroaryl or heteroaryloxy having 4 to 30 ring atoms which is unsubstituted or which is substituted by one or more halogen atoms or by one or more groups R¹⁰, —C(O)—R¹⁰, —C(O)—O—R¹⁰, or —O—C(O)—R¹⁰ as defined above,
R⁵ denotes —C(O)—R¹⁰, —C(O)—O—R¹⁰, or —O—C(O)—R¹⁰, wherein R¹⁰ is aryl or heteroaryl having 4 to 30 ring atoms which is unsubstituted or which is substituted by one or more halogen atoms or by one or more groups R¹ as defined above,
R³ and R⁴ denote H,
R³ and R⁴ are independently of each other selected from the group consisting of primary alkyl or alkoxy with 1 to 30 C atoms, secondary alkyl or alkoxy with 3 to 30 C atoms, and tertiary alkyl or alkoxy with 4 to 30 C atoms, wherein in all these groups one or more H atoms are optionally replaced by F,
R³ and R⁴ are independently of each other selected from the group consisting of aryl, heteroaryl, aryloxy, heteroaryloxy, each of which is optionally alkylated or alkoxylated and has 4 to 30 ring atoms,
R³ and/or R⁴ are independently of each other selected from the group consisting of alkyl, alkoxy, alkylcarbonyl, alkoxycarbonyl and alkylcarbonyloxy, all of which are straight-chain or branched, are optionally fluorinated, and have from 1 to 30 C atoms, and aryl, aryloxy, heteroaryl and heteroaryloxy, all of which are optionally alkylated or alkoxylated and have 4 to 30 ring atoms,
R³ and R⁴ denote independently of each other F, Cl, Br, I, CN, R¹⁰, —C(O)—R¹⁰, —C(O)—O—R¹⁰, or —O—C(O)—R¹⁰, wherein R¹⁰ is straight-chain, branched or cyclic alkyl with 1 to 30 C atoms, in which one or more non-adjacent C atoms are optionally replaced by —O—, —S—, —C(O)—, —C(O)—O—, —O—C(O)—, —O—C(O)—O—, —CR⁰═CR⁰⁰— or —C≡C— and in which one or more H atoms are optionally replaced by F, Cl, Br, I or CN, or R¹⁰ is aryl or heteroaryl having 4 to 30 ring atoms which is unsubstituted or which is substituted by one or more halogen atoms or by one or more groups R¹ as defined above,
R³ and/or R⁴ denote independently of each other aryl, aryloxy, heteroaryl or heteroaryloxy having 4 to 30 ring atoms which is unsubstituted or which is substituted by one or more halogen atoms or by one or more groups R¹⁰, —C(O)—R¹⁰, —C(O)—O—R¹⁰, or —O—C(O)—R¹⁰ as defined above,
R¹⁰ is primary alkyl with 1 to 30 C atoms, very preferably with 1 to 15 C atoms, secondary alkyl with 3 to 30 C atoms, or tertiary alkyl with 4 to 30 C atoms, wherein in all these groups one or more H atoms are optionally replaced by F,
R¹⁰ is aryl or heteroaryl having 4 to 30 ring atoms which is unsubstituted or which is substituted by one or more halogen atoms or by one or more groups R¹ as defined above,
R⁰ and R⁰⁰ are selected from H or C₁-C₁₀-alkyl,
R⁶ and R⁷ are selected from H, halogen, —CH₂Cl, —CHO, —CH═CH₂—SiR′R″R′″, —SnR′R″R′″, —BR′R″, —B(OR′)(OR″), —B(OH)₂, P-Sp, C₁-C₂₀-alkyl, C₁-C₂₀-alkoxy, C₂-C₂₀-alkenyl, C₁-C₂₀-fluoroalkyl and optionally substituted aryl or heteroaryl,
R⁸ and R⁹ are independently of each other selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z⁴)₂, —C≡CH and —Sn(Z⁴)₃, wherein Z¹⁻⁴ are selected from the group consisting of alkyl and aryl, each being optionally substituted, and two groups Z² may also form a cyclic group, very preferably from Br.

The polymers of the present invention can be synthesized according to or in analogy to methods that are known to the skilled person and are described in the literature. Other methods of preparation can be taken from the examples. For example, they can be suitably prepared by aryl-aryl coupling reactions, such as Yamamoto coupling, Suzuki coupling, Stille coupling, Sonogashira coupling, Heck coupling or Buchwald coupling. Suzuki coupling and Yamamoto coupling are especially preferred.

The monomers which are polymerised to form the repeat units of the polymers can be prepared according to methods which are known to the person skilled in the art.

Preferably the polymers are prepared from monomers of formula Ia or its preferred embodiments as described above and below.

Another aspect of the invention is a process for preparing a polymer by coupling one or more identical or different monomers of formula II with each other in a polymerisation reaction, preferably in an aryl-aryl coupling reaction.

Preferred methods for polymerisation are those leading to C—C-coupling or C—N-coupling, like Suzuki polymerisation, as described for example in WO 00/53656, Yamamoto polymerisation, as described in for example in T. Yamamoto et al., Progress in Polymer Science 1993, 17, 1153-1205 or in WO 2004/022626 A1, and Stille coupling. For example, when synthesizing a linear polymer by Yamamoto polymerisation, monomers as described above having two reactive halide groups R⁵ and R⁶ is preferably used. When synthesizing a linear polymer by Suzuki polymerisation, preferably a monomer as described above is used wherein at least one reactive group R⁵ or R⁶ is a boronic acid or boronic acid derivative group.

Suzuki polymerisation may be used to prepare homopolymers as well as statistical, alternating and block random copolymers. Statistical or block copolymers can be prepared for example from the above monomers of formula V wherein one of the reactive groups R⁵ and R⁶ is halogen and the other reactive group is a boronic acid or boronic acid derivative group. The synthesis of statistical, alternating and block copolymers is described in detail for example in WO 03/048225 A2 or WO 2005/014688 A2.

Suzuki polymerisation employs a Pd(0) complex or a Pd(II) salt. Preferred Pd(0) complexes are those bearing at least one phosphine ligand such as Pd(Ph₃P)₄. Another preferred phosphine ligand is tris(ortho-tolyl)phosphine, i.e. Pd(o-Tol)₄. Preferred Pd(II) salts include palladium acetate, i.e. Pd(OAc)₂. Suzuki polymerisation is performed in the presence of a base, for example sodium carbonate, potassium phosphate or an organic base such as tetraethylammonium carbonate. Yamamoto polymerisation employs a Ni(0) complex, for example bis(1,5-cyclooctadienyl) nickel(0).

As alternatives to halogens as described above, leaving groups of formula —O—SO₂Z¹ can be used wherein Z¹ is as described above. Particular examples of such leaving groups are tosylate, mesylate and triflate.

Especially suitable and preferred synthesis methods of the monomers and polymers of the present invention are illustrated in the synthesis schemes shown hereinafter, wherein R¹⁻⁵ are as defined above.

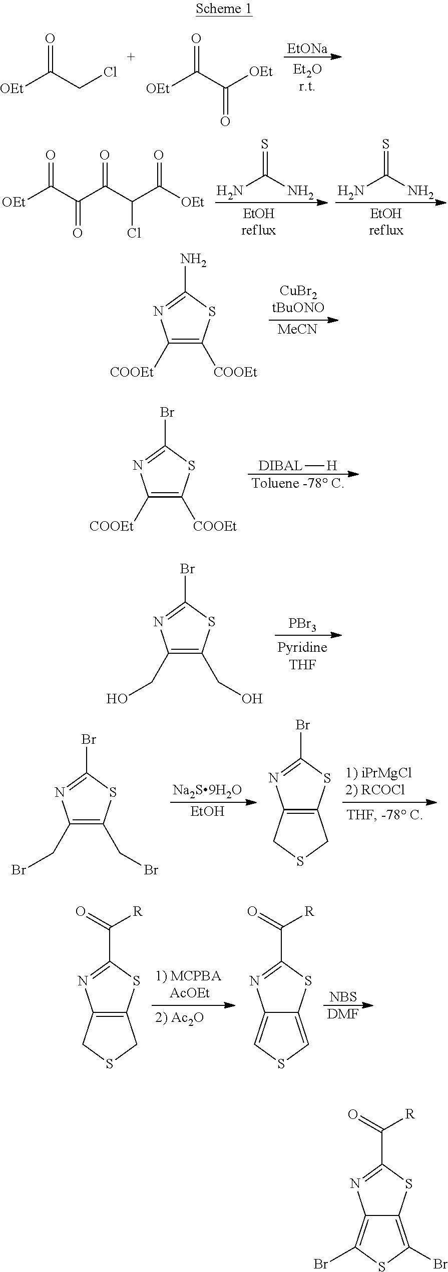

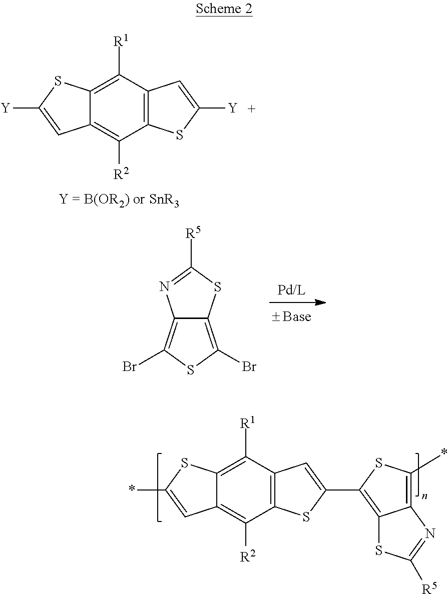

The novel methods of preparing monomers and polymers as described above and below are another aspect of the invention.

The polymers according to the present invention can also be used in mixtures or polymer blends, for example together with monomeric compounds or together with other polymers having charge-transport, semiconducting, electrically conducting, photoconducting and/or light emitting semiconducting properties, or for example with polymers having hole blocking or electron blocking properties for use as interlayers or charge blocking layers in OLED devices. Thus, another aspect of the invention relates to a polymer blend comprising one or more polymers according to the present invention and one or more further polymers having one or more of the above-mentioned properties. These blends can be prepared by conventional methods that are described in prior art and known to the skilled person. Typically the polymers are mixed with each other or dissolved in suitable solvents and the solutions combined.

Another aspect of the invention relates to a formulation comprising one or more polymers, mixtures or polmyer blends as described above and below and one or more organic solvents.

Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetramethyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, dimethylformamide, 2-chloro-6fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylanisole, 3-methylanisole, 4-fluoro-3-methylanisole, 2-fluorobenzonitrile, 4-fluoroveratrol, 2,6-dimethylanisole, 3-fluorobenzonitrile, 2,5-dimethylanisole, 2,4-dimethylanisole, benzonitrile, 3,5-dimethylanisole, N,N-dimethylaniline, ethyl benzoate, 1-fluoro-3,5-dimethoxybenzene, 1-methylnaphthalene, N-methylpyrrolidinone, 3-fluorobenzotrifluoride, benzotrifluoride, benzotrifluoride, diosane, trifluoromethoxybenzene, 4-fluorobenzotrifluoride, 3-fluoropyridine, toluene, 2-fluorotoluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluorobenzene, 3-chlorofluorobenzene, 1-chloro-2,5-difluorobenzene, 4-chlorofluorobenzene, chlorobenzene, o-dichlorobenzene, 2-chlorofluorobenzene, p-xylene, m-xylene, o-xylene or mixture of o-, m-, and p-isomers. Solvents with relatively low polarity are generally preferred. For inkjet printing solvents with high boiling temperatures and solvent mixtures are preferred. For spin coating alkylated benzenes like xylene and toluene are preferred.

Examples of especially preferred solvents include, without limitation, dichloromethane, trichloromethane, monochlorobenzene, o-dichlorobenzene, tetrahydrofuran, anisole, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, dimethylformamide, dimethylacetamide, dimethylsulfoxide, tetraline, decaline, indane, methyl benzoate, ethyl benzoate, mesitylene and/or mixtures thereof.

The concentration of the polymers in the solution is preferably 0.1 to 10% by weight, more preferably 0.5 to 5% by weight. Optionally, the solution also comprises one or more binders to adjust the rheological properties, as described for example in WO 2005/055248 A1.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 38, No 496, 296 (1966)”. Solvent blends may also be used and can be identified as described in “Solvents, W. H. Ellis, Federation of Societies for Coatings Technology, p 9-10, 1986”. Such a procedure may lead to a blend of ‘non’ solvents that will dissolve both the polymers of the present invention, although it is desirable to have at least one true solvent in a blend.

The polymers according to the present invention can also be used in patterned OSC layers in the devices as described above and below. For applications in modern microelectronics it is generally desirable to generate small structures or patterns to reduce cost (more devices/unit area), and power consumption. Patterning of thin layers comprising a polymer according to the present invention can be carried out for example by photolithography, electron beam lithography or laser patterning.

For use as thin layers in electronic or electrooptical devices the polymers, polymer blends or formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, letter-press printing, screen printing, doctor blade coating, roller printing, reverse-roller printing, offset lithography printing, flexographic printing, web printing, spray coating, brush coating or pad printing. Ink-jet printing is particularly preferred as it allows high resolution layers and devices to be prepared.

Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the polymers should be first dissolved in a suitable solvent. Solvents must fulfil the requirements stated above and must not have any detrimental effect on the chosen print head. Additionally, solvents should have boiling points >100° C., preferably >140° C. and more preferably >150° C. in order to prevent operability problems caused by the solution drying out inside the print head. Apart from the solvents mentioned above, suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a polymer according to the present invention by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the polymer, which reduces or prevents clogging of the jets and separation of the components during spraying. The solvent(s) may include those selected from the following list of examples: dodecylbenzene, 1-methyl-4-tert-butylbenzene, terpineol limonene, isodurene, terpinolene, cymene, diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1-100 mPa·s, more preferably 1-50 mPa·s and most preferably 1-30 mPa·s.

The polymers or formulations according to the present invention can additionally comprise one or more further components or additives selected for for example from surface-active compounds, lubricating agents, wetting agents, dispersing agents, hydrophobing agents, adhesive agents, flow improvers, defoaming agents, deaerators, diluents which may be reactive or non-reactive, auxiliaries, colourants, dyes or pigments, sensitizers, stabilizers, nanoparticles or inhibitors.

The polymers according to the present invention are useful as charge transport, semiconducting, electrically conducting, photoconducting or light mitting materials in optical, electrooptical, electronic, electroluminescent or photoluminescent components or devices. In these devices, the polymers of the present invention are typically applied as thin layers or films.

Thus, the present invention also provides the use of the semiconducting polymer, polymer blend, formulation or layer in an electronic device. The formulation may be used as a high mobility semiconducting material in various devices and apparatus. The formulation may be used, for example, in the form of a semiconducting layer or film. Accordingly, in another aspect, the present invention provides a semiconducting layer for use in an electronic device, the layer comprising a polymer, polymer blend or formulation according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The invention additionally provides an electronic device comprising a polymer, polymer blend, formulation or organic semiconducting layer according to the present invention. Especially preferred devices are OFETs, TFTs, ICs, logic circuits, capacitors, RFID tags, OLEDs, OLETs, OPEDs, OPVs, solar cells, laser diodes, photoconductors, photodetectors, electrophotographic devices, electrophotographic recording devices, organic memory devices, sensor devices, charge injection layers, Schottky diodes, planarising layers, antistatic films, conducting substrates and conducting patterns.

Especially preferred electronic device are OFETs, OLEDs and OPV devices, in particular bulk heterojunction (BHJ) OPV devices. In an OFET, for example, the active semiconductor channel between the drain and source may comprise the layer of the invention. As another example, in an OLED device, the charge (hole or electron) injection or transport layer may comprise the layer of the invention.

For use in OPV devices the compound or polymer according to the present invention is preferably used as photo-active layer. This implies the use in a formulation that comprises or contains, more preferably consists essentially of, very preferably exclusively of, a p-type (electron donor) semiconductor and an n-type (electron acceptor) semiconductor. The p-type semiconductor is constituted by a compound, preferably a polymer according to the present invention. The n-type semiconductor can be an inorganic material such as zinc oxide or cadmium selenide, or an organic material such as graphene or a fullerene or substituted fullerene, for example an indene-C₆₀-fullerene bisaduct like ICBA, or a (6,6)-phenyl-butyric acid methyl ester derivatized methano C₆₀ fullerene, also known as “PCBM” or “C₆₀PCBM”, as disclosed for example in G. Yu, J. Gao, J. C. Hummelen, F. Wudl, A. J. Heeger, Science, 1995, 270, 1789 and having the structure shown below, or structural analogous compounds with e.g. a C₆₁ fullerene group, a C₇₀ fullerene group, or a C₇₁ fullerene group, or an organic polymer (see for example Coakley, K. M. and McGehee, M. D. Chem. Mater., 2004, 16, 4533).

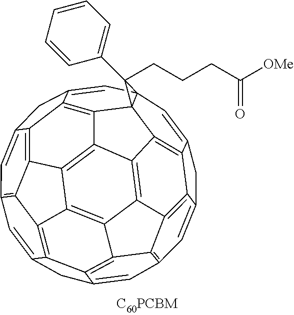 [SMILES: COC(=O)CCCC1(c2ccccc2)C23C=C4Cc5cc6cc7c8c9c(cc%10cc%11c%12c(c%13c%14c%15c(c4c5c4c6c8c(c%154)c4c9c%10c%12c%144)C%1321)C(=C3)C%11)C7]

Very preferred is a blend or mixture of a polymer according to the present invention with a C₆₀, C₆₁, C₇₀ or C₇₁ fullerene or substituted fullerene like C₆₀PCBM, C₆₁PCBM, C₇₀PCBM, C₇₁PCBM, bis-PCBM-C₆₁, bis-PCBM-C₇₁, graphene or ICBA. Preferably the ratio polymer:fullerene is from 2:1 to 1:2 by weight, more preferably from 1.2:1 to 1:1.2 by weight, most preferably 1:1 by weight. For the blended mixture, an optional annealing step may be necessary to optimize blend morpohology and consequently OPV device performance.

The OPV device can for example be of any type known from the literature (see for example Waldauf et al., Appl. Phys. Lett. 89, 233517 (2006), or Coakley, K. M. and McGehee, M. D. Chem. Mater. 2004, 16, 4533).

A first preferred OPV device according to the invention comprises the following layers (in the sequence from bottom to top):
a high work function electrode preferably comprising a metal oxide like for example ITO, serving as anode,
an optional conducting polymer layer or hole transport layer, preferably comprising an organic polymer or polymer blend, for example of PEDOT:PSS (poly(3,4-ethylenedioxythiophene): poly(styrene-sulfonate),
a layer, also referred to as “active layer”, comprising a p-type and an n-type organic semiconductor, which can exist for example as a p-type/n-type bilayer or as distinct p-type and n-type layers, or as blend or p-type and n-type semiconductor, forming a BHJ,
optionally a layer having electron transport properties, for example comprising LiF,
a low work function electrode, preferably comprising a metal like for example aluminum, serving as cathode,
wherein at least one of the electrodes, preferably the anode, is transparent to visible light, and
wherein the p-type semiconductor is a polymer according to the present invention.

A second preferred OPV device according to the invention is an inverted OPV device and comprises the following layers (in the sequence from bottom to top):
an electrode comprising for example ITO serving as cathode,
optionally a layer having hole blocking properties, preferably comprising a metal oxide like TiOₓ or Znₓ,
an active layer comprising a p-type and an n-type organic semiconductor, situated between the electrodes, which can exist for example as a p-type/n-type bilayer or as distinct p-type and n-type layers, or as blend or p-type and n-type semiconductor, forming a BHJ,
an optional conducting polymer layer or hole transport layer, preferably comprising an organic polymer or polymer blend, for example of PEDOT:PSS,
a high work function electrode, preferably comprising a metal like for example gold, serving as anode,
wherein at least one of the electrodes, preferably the cathode, is transparent to visible light, and
wherein the p-type semiconductor is a polymer according to the present invention.

In the OPV devices of the present invent invention the p-type and n-type semiconductor materials are preferably selected from the materials, like the polymer/fullerene systems, as described above. If the bilayer is a blend an optional annealing step may be necessary to optimize device performance.

The compound, formulation and layer of the present invention are also suitable for use in an OFET as the semiconducting channel. Accordingly, the invention also provides an OFET comprising a gate electrode, an insulating (or gate insulator) layer, a source electrode, a drain electrode and an organic semiconducting channel connecting the source and drain electrodes, wherein the organic semiconducting channel comprises a polymer, polymer blend, formulation or organic semiconducting layer according to the present invention. Other features of the OFET are well known to those skilled in the art.

OFETs where an OSC material is arranged as a thin film between a gate dielectric and a drain and a source electrode, are generally known, and are described for example in U.S. Pat. No. 5,892,244, U.S. Pat. No. 5,998,804, U.S. Pat. No. 6,723,394 and in the references cited in the background section. Due to the advantages, like low cost production using the solubility properties of the compounds according to the invention and thus the processibility of large surfaces, preferred applications of these FETs are such as integrated circuitry, TFT displays and security applications.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers,
optionally a substrate.
   wherein the semiconductor layer preferably comprises a polymer, polymer blend or formulation as described above and below.

The OFET device can be a top gate device or a bottom gate device. Suitable structures and manufacturing methods of an OFET device are known to the skilled in the art and are described in the literature, for example in US 2007/0102696 A1.

The gate insulator layer preferably comprises a fluoropolymer, like e.g. the commercially available Cytop 809M® or Cytop 107M® (from Asahi Glass). Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377). Especially preferred are organic dielectric materials having a low permittivity (or dielectric constant) from 1.0 to 5.0, very preferably from 1.8 to 4.0 (“low k materials”), as disclosed for example in US 2007/0102696 A1 or U.S. Pat. No. 7,095,044.

In security applications, OFETs and other devices with semiconducting materials according to the present invention, like transistors or diodes, can be used for RFID tags or security markings to authenticate and prevent counterfeiting of documents of value like banknotes, credit cards or ID cards, national ID documents, licenses or any product with monetry value, like stamps, tickets, shares, cheques etc.

Alternatively, the materials according to the invention can be used in OLEDs, e.g. as the active display material in a flat panel display applications, or as backlight of a flat panel display like e.g. a liquid crystal display. Common OLEDs are realized using multilayer structures. An emission layer is generally sandwiched between one or more electron-transport and/or hole-transport layers. By applying an electric voltage electrons and holes as charge carriers move towards the emission layer where their recombination leads to the excitation and hence luminescence of the lumophor units contained in the emission layer. The inventive compounds, materials and films may be employed in one or more of the charge transport layers and/or in the emission layer, corresponding to their electrical and/or optical properties. Furthermore their use within the emission layer is especially advantageous, if the compounds, materials and films according to the invention show electroluminescent properties themselves or comprise electroluminescent groups or compounds. The selection, characterization as well as the processing of suitable monomeric, oligomeric and polymeric compounds or materials for the use in OLEDs is generally known by a person skilled in the art, see, e.g., Meerholz, Synthetic Materials, 111-112, 2000, 31-34, Alcala, J. Appl. Phys., 88, 2000, 7124-7128 and the literature cited therein.

According to another use, the materials according to this invention, especially those showing photoluminescent properties, may be employed as materials of light sources, e.g. in display devices, as described in EP 0 889 350 A1 or by C. Weder et al., Science, 279, 1998, 835-837.

A further aspect of the invention relates to both the oxidised and reduced form of the compounds according to this invention. Either loss or gain of electrons results in formation of a highly delocalised ionic form, which is of high conductivity. This can occur on exposure to common dopants. Suitable dopants and methods of doping are known to those skilled in the art, e.g. from EP 0 528 662, U.S. Pat. No. 5,198,153 or WO 96/21659.

The doping process typically implies treatment of the semiconductor material with an oxidating or reducing agent in a redox reaction to form delocalised ionic centres in the material, with the corresponding counterions derived from the applied dopants. Suitable doping methods comprise for example exposure to a doping vapor in the atmospheric pressure or at a reduced pressure, electrochemical doping in a solution containing a dopant, bringing a dopant into contact with the semiconductor material to be thermally diffused, and ion-implantantion of the dopant into the semiconductor material.

When electrons are used as carriers, suitable dopants are for example halogens (e.g., I₂, Cl₂, Br₂, ICl, ICl₃, IBr and IF), Lewis acids (e.g., PF₅, AsF₅, SbF₅, BF₃, BCl₃, SbCl₅, BBr₃ and SO₃), protonic acids, organic acids, or amino acids (e.g., HF, HCl, HNO₃, H₂SO₄, HClO₄, FSO₃H and ClSO₃H), transition metal compounds (e.g., FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid), anions (e.g., Cl⁻, Br⁻, I⁻, I₃⁻, HSO₄⁻, SO₄²⁻, NO₃⁻, ClO₄⁻, BF₄⁻, PF₆⁻, AsF₆⁻, SbF₆⁻, FeCl₄⁻, Fe(CN)₆³⁻, and anions of various sulfonic acids, such as aryl-SO₃⁻). When holes are used as carriers, examples of dopants are cations (e.g., H⁺, Li⁺, Na⁺, K⁺, Rb⁺ and Cs⁺), alkali metals (e.g., Li, Na, K, Rb, and Cs), alkaline-earth metals (e.g., Ca, Sr, and Ba), O₂, XeOF₄, (NO₂⁺)(SbF₆⁻), (NO₂⁺) (SbCl₆⁻), (NO₂⁺)(BF₄⁻), AgClO₄, H₂IrCl₆, La(NO₃)₃.6H₂O, FSO₂OOSO₂F, Eu, acetylcholine, R₄N⁺, (R is an alkyl group), R₄P⁺ (R is an alkyl group), R₆As⁺ (R is an alkyl group), and R₃S⁺ (R is an alkyl group).

The conducting form of the compounds of the present invention can be used as an organic “metal” in applications including, but not limited to, charge injection layers and ITO planarising layers in OLED applications, films for flat panel displays and touch screens, antistatic films, printed conductive substrates, patterns or tracts in electronic applications such as printed circuit boards and condensers.

The compounds and formulations according to the present invention may also be suitable for use in organic plasmon-emitting diodes (OPEDs), as described for example in Koller et al., Nature Photonics 2008 (published online Sep. 28, 2008).

According to another use, the materials according to the present invention can be used alone or together with other materials in or as alignment layers in LCD or OLED devices, as described for example in US 2003/0021913. The use of charge transport compounds according to the present invention can increase the electrical conductivity of the alignment layer. When used in an LCD, this increased electrical conductivity can reduce adverse residual dc effects in the switchable LCD cell and suppress image sticking or, for example in ferroelectric LCDs, reduce the residual charge produced by the switching of the spontaneous polarisation charge of the ferroelectric LCs. When used in an OLED device comprising a light emitting material provided onto the alignment layer, this increased electrical conductivity can enhance the electroluminescence of the light emitting material. The compounds or materials according to the present invention having mesogenic or liquid crystalline properties can form oriented anisotropic films as described above, which are especially useful as alignment layers to induce or enhance alignment in a liquid crystal medium provided onto said anisotropic film. The materials according to the present invention may also be combined with photoisomerisable compounds and/or chromophores for use in or as photoalignment layers, as described in US 2003/0021913.

According to another use the materials according to the present invention, especially their water-soluble derivatives (for example with polar or ionic side groups) or ionically doped forms, can be employed as chemical sensors or materials for detecting and discriminating DNA sequences. Such uses are described for example in L. Chen, D. W. McBranch, H. Wang, R. Helgeson, F. Wudl and D. G. Whitten, Proc. Natl. Acad. Sci. U.S.A. 1999, 96, 12287; D. Wang, X. Gong, P. S. Heeger, F. Rininsland, G. C. Bazan and A. J. Heeger, Proc. Natl. Acad. Sci. U.S.A. 2002, 99, 49; N. DiCesare, M. R. Pinot, K. S. Schanze and J. R. Lakowicz, Langmuir 2002, 18, 7785; D. T. McQuade, A. E. Pullen, T. M. Swager, Chem. Rev. 2000, 100, 2537.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

Above and below, unless stated otherwise percentages are percent by weight and temperatures are given in degrees Celsius. The values of the dielectric constant ∈ (“permittivity”) refer to values taken at 20° C. and 1,000 Hz.

The invention will now be described in more detail by reference to the following examples, which are illustrative only and do not limit the scope of the invention.

### Example 1

The synthesis of 2-amino-thiazole-4,5-dicarboxylic acid diethyl ester has been described, for example, in WO 2006/087543 A1.

### 2-Bromo-thiazole-4,5-dicarboxylic acid diethyl ester (1.1)

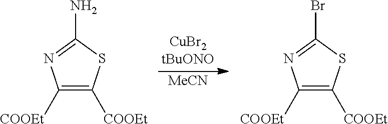 [SMILES: "MeCN" ; CCOC(=O)c1nc(N)sc1C(=O)OCC ; CCOC(=O)c1nc(Br)sc1C(=O)OCC]

t-Butyl nitrite (50.4 cm³; 424 mmol; 1.50 eq.) and copper bromide (94.64 g; 423.7 mmol; 1.500 eq.) are dissolved into acetonitrile (750 cm³). 2-Amino-thiazole-4,5-dicarboxylic acid diethyl ester (69.00 g; 282.5 mmol; 1.000 eq.) is added in one portion at 23° C. (note: gas evolution and heat generation). After 60 minutes, the resulting mixture is poured into a saturated sodium thiosulfate solution, acidified with 1M aqueous hydrochloriic acid and extracted with dichloromethane (3×250 cm³). The combined organic fraction are combined, dried over magnesium sulfate and removed in vacuo. The product (56.26 g, Yield: 65%) is used without further purification. NMR (¹H, 300 MHz, CDCl₃): δ 4.44 (q, J=7.1 Hz, 2H); 4.37 (q, J=7.1 Hz, 2H); 1.41 (t, J=7.1 Hz, 3H); 1.36 (t, J=7.1 Hz, 3H).

### (2-Bromo-5-hydroxymethyl-thiazol-4-yl)-methanol (1.2)

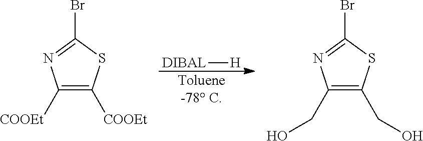

To a solution of 2-Bromo-thiazole-4,5-dicarboxylic acid diethyl ester (56.00 g; 181.7 mmol; 1.000 eq.) in toluene (725 cm³), a 1.0 M solution of DIBAL-H in toluene (545 cm³; 545 mmol; 3.00 eq.) is added dropwise over 60 minutes while stirring at −78° C. under nitrogen. The resulting mixture is kept for 3 h at −78° C. before adding with 60 cm³ of methanol and a saturated aqueous Rochelle's salt solution (500 cm³). The biphasic mixture is rapidly stirred for 18 hours at 23° C. whereupon two clear, colorless layers formed. The aqueous layer is withdrawn and extracted with dichloromethane (2×200 cm³). The combined organic phases are discarded. The aqueous phase is further extracted with diethyl ether multiple times to afford 16.01 g of the title compound. The water from aqueous phase is removed in vacuo and the residue washed in a Soxhlet apparatus with diethyl ether for 24 hours to afford an additional 4.05 g of the title compound. (Combined Yield: 49%). NMR (¹H, 300 MHz, Acetone-d₆): δ 4.88 (s, 2H); 4.85 (br, 1H); 4.63 (s, 2H); 4.34 (br, 1H).

### 2-Bromo-4,5-bis-bromomethyl-thiazole (1.3)

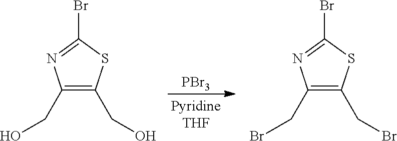 [SMILES: OCc1nc(Br)sc1CO ; "PBr3" ; BrCc1nc(Br)sc1CBr]

To a solution of (2-bromo-5-hydroxymethyl-thiazol-4-yl)-methanol (19.30 g; 86.131 mmol; 1.000 eq.) in anhydrous tetrahydrofuran (350 cm³), pyridine (7.0 cm³; 86 mmol; 1.0 eq.) is added dropwise with stirring at 0° C. under nitrogen. This mixture is kept for 15 minutes at 0° C. Phosphorus tribromide (16.2 cm³; 172 mmol; 2.00 eq.) is slowly added to the reaction at 0° C. The final mixture is kept for 1 hour at 0° C. and for 6 hours at 23° C. The crude is neutralized by adding saturated sodium bicarbonate in ice water (100 cm³) and the resulting mixture extracted with dichloromethane (2×500 cm³). The combined organic phase are washed with water, dried with magnesium sulfate and removed in vacuo. The resulting oil is purified by column chromatography using a gradient of petroleum ether and acetone (100:0 to 75:25) to afford 14.58 g of the title product as an oil which crystallized upon standing (Yield: 49%). NMR (¹H, 300 MHz, CDCl₃): δ 4.62 (s, 2H); 4.51 (s, 2H).

### 2-Bromo-4,6-dihydro-thieno[3,4-d]thiazole (1.4)

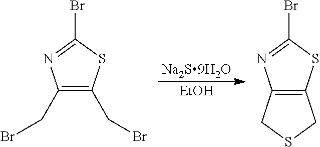 [SMILES: "EtOH" ; BrCc1nc(Br)sc1CBr ; Brc1nc2c(s1)CSC2]

A solution of 2-bromo-4,5-bis-bromomethyl-thiazole (10.50 g; 30.01 mmol; 1.000 eq.) in ethanol (210 cm³) is cooled down to 0° C. Sodium sulfide nonahydrate (7.208 g; 30.01 mmol; 1.000 eq.) is dissolved in ethanol (590 cm³) (note: gentle heating is required to completely dissolved the sodium sulfide nonahydrate) and added dropwise to the previous solution at 0° C. over 1 hour. After the addition is complete, the reaction mixture is stirred at 0° C. for additional hour and at 23° C. for 18 hours. The white precipitate is filtered off, discarded and the solvent removed in vacuo. The resulting solid is dissolved back in boiling ethanol (75 cm³) and the resulting insoluble white solid removed by filtration. The solution is allowed to cool down and the precipitate filtered off and discarded. The filtrate solvent is removed in vacuo to afford the title product (1.225 g, Yield: 18%) as an off white solid. NMR (¹H, 300 MHz, CDCl₃): δ 4.12 (m, 4H).

### 1-(4,6-Dihydro-thieno[3,4-d]thiazol-2-yl)-2-ethyl-hexan-1-one (1.5)

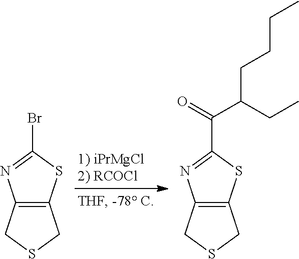 [SMILES: Brc1nc2c(s1)CSC2 ; CCCCC(CC)C(=O)c1nc2c(s1)CSC2]

The 2-bromo-4,6-dihydro-thieno[3,4-d]thiazole (0.800 g; 3.60 mmol; 1.00 eq.) is dissolved in anhydrous tetrahydrofuran (36 cm³) and cooled down to −78° C. A 2.0 M solution of isopropylmagnesium chloride in tetrahydrofuran (2.0 ml; 4.0 mmol; 1.1 eq.) is added dropwise over 5-10 minutes and the resulting mixture stirred at −78° C. for 20 minutes and at 0° C. for 20 minutes. This solution is transferred into a dropping funnel kept at 0° C. and added dropwise over 5-10 minutes to a solution of 2-ethyl-hexanoyl chloride (0.78 cm³; 4.5 mmol; 1.25 eq.) in anhydrous tetrahydrofuran (36 cm³) at −78° C. After 30 minutes, the reaction mixture is poured into water and extracted with dichloromethane (3×50 cm³). The combined organic layer are washed with water (100 cm³) before been dried over magnesium sulfate and the solvent removed in vacuo. The recovered crude product is purified by column chromatography using a gradient of petroleum ether and dichloromethane (20:80 to 0:100) to afford 0.465 g of the title product (Yield: 48%). NMR (¹H, 300 MHz, CDCl₃): δ 4.20 (s, 4H); 3.62 (m, 1H); 1.78 (m, 2H); 1.62 (m, 2H); 1.25 (m, 4H); 0.89 (t, J=7.4 Hz, 3H); 0.86 (t, J=7.0 Hz, 3H).

### 2-Ethyl-1-thieno[3,4-d]thiazol-2-yl-hexan-1-one (1.6)

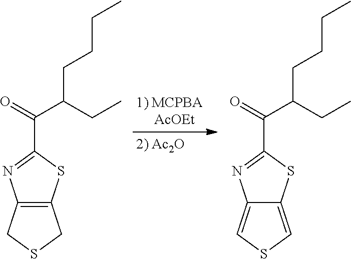 [SMILES: CCCCC(CC)C(=O)c1nc2c(s1)CSC2 ; CCCCC(CC)C(=O)c1nc2cscc2s1]

1-(4,6-Dihydro-thieno[3,4-d]thiazol-2-yl)-2-ethyl-hexan-1-one (0.300 g; 1.113 mmol; 1.00 eq.) is dissolved in ethyl acetate (22 cm³) and cooled down to −78° C. 3-chloro-benzenecarboperoxoic acid (MCPBA) (0.192 g; 1.11 mmol; 1.000 eq.) in ethyl acetate (11 cm³) is added dropwise over 5-10 minutes. The resulting mixture is stirred at −78° C. for 1 hour and at 23° C. for 18 hours. The solvent is removed in vacuo and the solid containing crude sulfinyl and the residual MCPBA is refluxed in acetic anhydride (22 cm³) for 2.5 hours. The residual solvent is removed in vacuo and the recovered crude product purified by column chromatography using petroleum ether and dichloromethane (50:50) to afford the title product as a yellow oil (0.206 g, Yield: 69%). NMR (¹H, 300 MHz, CDCl₃): δ 7.93 (d, J=2.8 Hz, 2H); 7.36 (d, J=2.8 Hz, 2H); 3.75 (m, 1H); 1.83 (m, 2H); 1.67 (m, 2H); 1.28 (m, 4H); 0.92 (t, J=7.4 Hz, 3H); 0.86 (t, J=7.0 Hz, 3H).

### 1-(4,6-Dibromo-thieno[3,4-d]thiazol-2-yl)-2-ethyl-hexan-1-one (1.7)

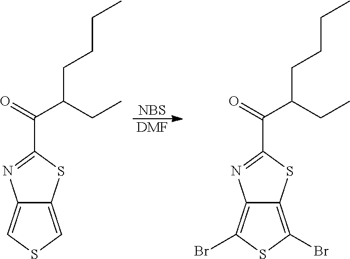

2-Ethyl-1-thieno[3,4-d]thiazol-2-yl-hexan-1-one (0.275 g; 1.03 mmol; 1.00 eq.) is dissolved in anhydrous N,N-dimethylformamide (3.00 cm³). Under the protection of inert atmosphere, 1-bromo-pyrrolidine-2,5-dione (NBS) (0.458 g; 2.57 mmol; 2.50 eq.) is added in one portion. The reactants are stirred for 20 minutes and then poured into 10 cm³ of 5% sodium thiosulfate solution. The mixture is extracted several times by diethyl ether. The combined organic phases are dried over sodium sulfate and removed in vacuo. The recovered crude product is purified by column chromatography using petroleum ether and dichloromethane (50:50) to afford the title product (0.332 g) as a reddish oil which crystallized upon standing (Yield: 76%). NMR (¹H, 300 MHz, CDCl₃): δ 3.73 (m, 1H); 1.81 (m, 2H); 1.67 (m, 2H); 1.29 (m, 4H); 0.91 (t, J=7.4 Hz, 3H); 0.87 (t, J=7.0 Hz, 3H).

### Poly(4,8-dioctyl-benzo[1,2-b;4,5-b′]dithiophene-2,6-diyl-alt-2-(2-ethyl-hexan-1-one)-thieno[3,4-d]thiazole-4,6-diyl) (1.8)

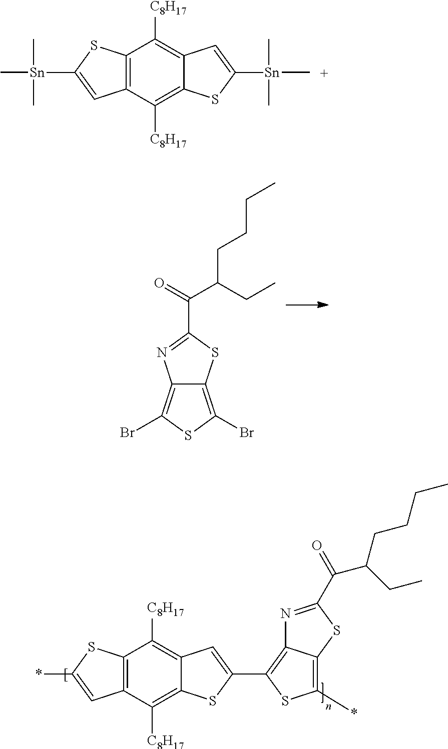

1-(4,6-Dibromo-thieno[3,4-d]thiazol-2-yl)-2-ethyl-hexan-1-one (251.0 mg; 0.5903 mmol; 1.000 eq.) is weighted into a 20 cm³ microwave vial and then 4,8-dioctyl-2,6-bis-trimethylstannanyl-benzo[1,2-b;4,5-b′]dithiophene (437.0 mg; 0.5903 mmol; 1.000 eq.), tri-o-tolyl-phosphine (14.4 mg; 0.0472 mmol; 0.080 eq.) and tris(dibenzylideneacetone)dipalladium(0) (5.4 mg; 0.0059 mmol; 0.010 eq.) are added. The flask is subjected to three successive cycles of vacuum followed by refilling with nitrogen. Then, anhydrous degassed N,N′-dimethylformamide (1.6 cm³) and anhydrous degassed Toluene (10 cm³) are added via a syringe. The reaction is heated over microware (Initiator, Biotage AB) at 120° C. for 2 minutes, 140° C. for 2 minutes, 160° C. for 2 minutes and 170° C. for 20 minutes. The polymer was purified by precipitation into methanol, filtered and washed sequentially via Soxhlet extraction with acetone, petroleum ether (40-60° C.), cyclohexane. The cyclohexane fraction was reduced to a smaller volume and precipitated into methanol (200 cm³). The precipitated polymer was filtered and dried under vacuum at 25° C. overnight to afford the product (215 mg, yield 54%). GPC (Chlorobenzene, 50° C.): Mₙ=13.2 kg.mol⁻¹, M_{w}=36.5 kg.mol⁻¹.

### Example 2

### Poly(4,8-dioctyl-benzo[1,2-b;4,5-b′]dithiophene-2,6-diyl-alt-2-(2-ethyl-hexan-1-one)-thieno[3,4-d]thiazole-4,6-diyl) (2.1)

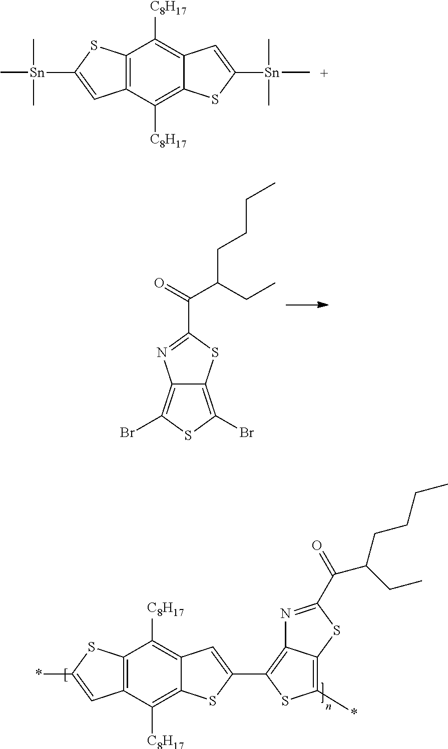

1-(4,6-Dibromo-thieno[3,4-d]thiazol-2-yl)-2-ethyl-hexan-1-one (310.8 mg; 730.9 μmol; 1.000 eq.) is weighted into a 20 cm³ microwave vial and then 4,8-dioctyl-2,6-bis-trimethylstannanyl-benzo[1,2-b;4,5-b′]dithiophene (541.1 mg; 730.9 μmol; 1.000 eq.), tri-o-tolyl-phosphine (18.0 mg; 58.5 μmol; 0.0800 eq.) and tris(dibenzylideneacetone)dipalladium(0) (13.4 mg; 14.6 μmol; 0.0200 eq.) are added. The flask is subjected to three successive cycles of vacuum followed by refilling with nitrogen. Then, anhydrous degassed chlorobenzene (7.3 cm³) is added via a syringe. The reaction is heated over microware (Initiator, Biotage AB) at 140° C. for 1 minute, 160° C. for 1 minute and 180° C. for 30 minutes. Immediately after completion of the reaction, the reaction was allowed to cool to 65° C., tributyl-phenyl-stannane (0.24 cm³; 0.73 mmol; 1.0 eq.) is added and the mixture heated back to 180° C. for 10 minutes. Immediately after completion of the first end-capping reaction, the reaction was allowed to cool to 65° C., bromobenzene (0.12 cm³; 1.1 mmol; 1.5 eq.) is added and the mixture heated back to 180° C. for 10 minutes. After completion of the second end-capping reaction, the mixture is allowed to cool to 65° C. and precipitated into stirred methanol (100 cm³) with methanol washings (2×10 cm³) of the reaction tube. The polymer was purified by precipitation into methanol, filtered and washed sequentially via Soxhlet extraction with acetone, petroleum ether (40-60° C.), cyclohexane and chloroform. Methanol (200 cm³) is dropwise to the chloroform fraction (150 cm³), the precipitated polymer filtered and dried under vacuum to afford the product (465 mg, yield 94%). GPC (Chlorobenzene, 50° C.): Mₙ=55.1 kg.mol⁻¹, M_{w}=111.9 kg.mol⁻¹.

### Example 3

OPV devices are fabricated on ITO-glass substrates (13Ω/), purchased from Zencatec. The substrates are subjected to a conventional photolithography process to define the bottom electrodes (anodes) before cleaning using common solvents (acetone, IPA, DI water) in an ultrasonic bath.

A conducting polymer poly(ethylene dioxythiophene) doped with poly(styrene sulfonic acid) [Clevios VPAI 4083 (H. C. Starck)] is mixed in a 1:1 ratio with DI-water. This solution is sonicated for 20 minutes to ensure proper mixing and filtered using a 0.2 μm filter before spin coating to a thickness of 20 nm. The substrates are exposed to a UV-ozone treatment prior to the spin-coating process to ensure good wetting properties. The films are then annealed at 130° C. for 30 minutes in an inert atmosphere.

Photoactive material solutions are prepared at the concentration and components ratio stated in Table 1 below, and stirred overnight. Thin films are either spin coated or blade coated in an inert atmosphere to achieve thicknesses between 100 and 200 nm, measured using a profilemeter. A short drying period follows to ensure removal of excess solvent. Spin coated films are dried at 23° C. for 10 minutes. Blade coated films are dried at 70° C. for 3 minutes on the hotplate.

As the last step of the device fabrication, Calcium (30 nm)/Al (200 nm) cathodes are thermally evaporated through a shadow mask to define cells. Samples are measured at 23° C. using a Solar Simulator from Newport Ltd (model 91160) as a light source, calibrated to 1 sun using a Si reference cell.

The device performance for the polymers of Example 1 and Example 2 is described in Table 1.

TABLE 1
Average open circuit potential (V_{oc}), current density (J_{SC}), fill
factor (FF), power conversion efficiency (PCE) and best power conversion
efficiency for specific ratios of PCBM-C₆₀ ratio and the polymer of
Example 1 or 2, respectively.

| | ratio | concⁿ | Voc | Jsc | FF | PCE | | Best PCE |
|---|---|---|---|---|---|---|---|---|
| | Polymer:PCBM | mg · ml⁻¹ | mV | mA · cm⁻² | % | % | PCE SD | % |
| Ex. 1 | 1.0:1.0 | 30 | 707 | −0.80 | 45.4 | 0.26 | 0.78 | 0.33 |
| | 1.0:1.5 | 30 | 730 | −1.02 | 47.9 | 0.36 | 0.82 | 0.44 |
| | 1.0:2.0 | 30 | 731 | −0.76 | 49.6 | 0.27 | 0.89 | 0.31 |
| Ex. 2 | 1.0:1.0 | 30 | 702 | −0.85 | 35.5 | 0.21 | 0.05 | 0.70 |
| | 1.0:1.5 | 30 | 708 | −1.23 | 43.1 | 0.38 | 0.10 | 0.62 |
| | 1.0:2.0 | 30 | 724 | −1.97 | 55.8 | 0.80 | 0.07 | 0.86 |
| | 1.0:3.0 | 30 | 712 | −1.71 | 53.3 | 0.65 | 0.11 | 0.81 |

1. A polymer of formula I 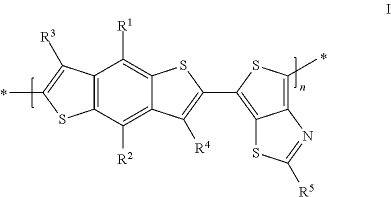
wherein
R¹ to R⁵ denote independently of each other, and on each occurrence identically or differently, H, halogen, or an optionally substituted carbyl or hydrocarbyl group, wherein one or more C atoms are optionally replaced by a hetero atom.

2. The polymer according to claim 1, wherein R¹, R², R³, R⁴ and R⁵ denote independently of each other F, Br, Cl, —CN, —NC, —NCO, —NCS, —OCN, —SCN, —C(O)NR⁰R⁰⁰, C(O)X⁰, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms, or P-Sp-, wherein
R⁰ and R⁰⁰ are independently of each other H or optionally substituted C₁₋₄₀ carbyl or hydrocarbyl,
P is a polymerisable or crosslinkable group,
Sp is a spacer group or a single bond,
X⁰ is F, Cl or Br.

3. The polymer according to claim 1, wherein R¹, R² and R⁵ denote independently of each other, and on each occurrence identically or differently, straight-chain, branched or cyclic alkyl with 1 to 30 C atoms, in which one or more non-adjacent C atoms are optionally replaced by —O—, —S—, —C(O)—, —C(O)—O—, —O—C(O)—, —CH═CH— or —C≡C— and which are unsubstituted or substituted by F, Cl, Br, I or CN.

4. The polymer according to claim 3, wherein R¹ and R² are independently of each other selected from the group consisting of alkyl, alkoxy, alkylcarbonyl, alkoxycarbonyl and alkylcarbonyloxy, all of which are straight-chain or branched, are optionally fluorinated, and have from 1 to 30 C atoms.

5. The polymer according to claim 1, wherein R³ and R⁴ are H.

6. The polymer according to claim 1, wherein R⁵ denotes F, Cl, Br, I, CN, R¹⁰, —C(O)—R¹⁰, —C(O)—O—R¹⁰, or —O—C(O)—R¹⁰, wherein R¹⁰ is straight-chain, branched or cyclic alkyl with 1 to 30 C atoms, in which one or more non-adjacent C atoms are optionally replaced by —O—, —S—, —C(O)—, —C(O)—O—, —O—C(O)—, —O—C(O)—O—, —CR⁰═CR⁰⁰— or —C≡C— and in which one or more H atoms are optionally replaced by F, Cl, Br, I or CN, or R¹⁰ is aryl or heteroaryl having 4 to 30 ring atoms which is unsubstituted or which is substituted by one or more halogen atoms or by one or more optionally substituted carbyl or hydrocarbyl group, wherein one or more C atoms are optionally replaced by a hetero atom.

7. The polymer according to claim 1, which is of formula II 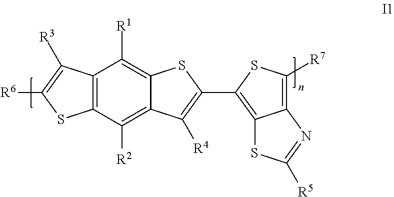
wherein
R¹⁻⁵ and n are as defined for the compound of formula I, and
R⁶ and R⁷ denote independently of each other, and on each occurrence identically or differently, H, halogen, or an optionally substituted carbyl or hydrocarbyl group, wherein one or more C atoms are optionally replaced by a hetero atom, or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′, P-Sp- or an endcap group, wherein P is a polymerisable or crosslinkable group, Sp is a spacer group or a single bond, X′ and X″ denote halogen, R′, R″ and R′″ are independently of each other H or optionally substituted C₁₋₄₀ carbyl or hydrocarbyl, and two of R′, R″ and R′″ may also form a ring together with the hetero atom to which they are attached

8. A monomer of formula II 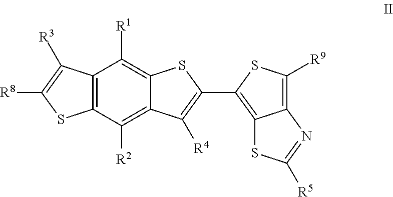
wherein R¹⁻⁵ denote independently of each other, and on each occurrence identically or differently, H, halogen, or an optionally substituted carbyl or hydrocarbyl group, wherein one or more C atoms are optionally replaced by a hetero atom, and R⁸ and R⁹ are independently of each other selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, Z¹⁻⁴ are selected from the group consisting of alkyl and aryl, each being optionally substituted, and two groups Z² may also together form a cyclic group.

9. A mixture or polymer blend comprising one or more polymers according to claim 1 and one or more compounds or polymers having semiconducting, charge transport, hole/electron transport, hole/electron blocking, electrically conducting, photoconducting or light emitting properties.

10. The mixture or polymer blend according to claim 9, which comprises one or more n-type organic semiconductor compounds.

11. The mixture or polymer blend according to claim 10, wherein the n-type organic semiconductor compound is selected from the group consisting of fullerenes, substituted fullerenes and graphene.

12. The mixture or polymer blend according to claim 11, wherein the n-type organic semiconductor compound is selected from the group consisting of PCBM-C₆₀, PCBM-C₇₀, PCBM-C₆₁, PCBM-C₇₁, bis-PCBM-C₆₁, bis-PCBM-C₇₁, ICBA and graphene.

13. A formulation comprising one or more polymers according to claim 1, and one or more solvents or organic solvents.

14. A charge transport, semiconducting, electrically conducting, photoconducting or light emitting material in an optical, electrooptical, electronic, electroluminescent or photoluminescent component or device, which comprises one or more polymers according to claim 1.

15. An optical, electrooptical or electronic component or device comprising one or more polymers according to claim 1.

16. A component or device according to claim 14, which is selected from the group consisting of organic field effect transistors (OFET), thin film transistors (TFT), integrated circuits (IC), logic circuits, capacitors, radio frequency identification (RFID) tags, devices or components, organic light emitting diodes (OLED), organic light emitting transistors (OLET), flat panel displays, backlights of displays, organic photovoltaic devices (OPV), organic solar cells (O-SC), photodiodes, laser diodes, photoconductors, photodetectors, electrophotographic devices, electrophotographic recording devices, organic memory devices, sensor devices, charge injection layers, charge transport layers or interlayers in polymer light emitting diodes (PLEDs), Schottky diodes, planarising layers, antistatic films, polymer electrolyte membranes (PEM), conducting substrates, conducting patterns, electrode materials in batteries, alignment layers, biosensors, biochips, security markings, security devices, and components or devices for detecting and discriminating DNA sequences.

17. A component or device according to claim 15, which is an OFET, bulk heterojunction (BHJ) OPV device, inverted BHJ OPV device or organic photodetector (OPD).

18. A process of preparing a polymer according to claim 1, comprising coupling one or more monomers with each other in an aryl-aryl coupling reaction, wherein said monomers are of formula II 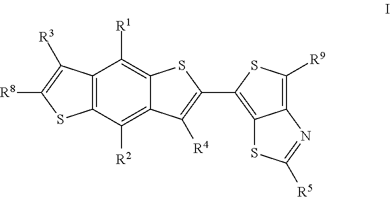
wherein R¹⁻⁵ denote independently of each other, and on each occurrence identically or differently, H, halogen, or an optionally substituted carbyl or hydrocarbyl group, wherein one or more C atoms are optionally replaced by a hetero atom, and R⁸ and R⁹ are independently of each other selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, Z¹⁻⁴ are selected from the group consisting of alkyl and aryl, each being optionally substituted, and two groups Z² may also together form a cyclic group.

19. The polymer according to claim 1, wherein R¹, R², R³, R⁴ and R⁵ denote independently of each other F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl with 1 to 40 C atoms that is optionally substituted and optionally comprises one or more hetero atoms, or P-Sp-, wherein
R⁰ and R⁰⁰ are independently of each other H,
P is a polymerisable or crosslinkable group,
Sp is a spacer group or a single bond,
X⁰ is F, Cl or Br.

20. A monomer according to claim 8, wherein R¹, R², R³, R⁴ and R⁵ denote independently of each other F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl with 1 to 40 C atoms that is optionally substituted and optionally comprises one or more hetero atoms, or P-Sp-, wherein
R⁰ and R⁰⁰ are independently of each other H,
P is a polymerisable or crosslinkable group,
Sp is a spacer group or a single bond,
X⁰ is F, Cl or Br.
